(19) United States
(12) Patent Application Publication
(10) Pub. No.: US 2003/0092637 A1
(43) Pub. Date: May 15, 2003

(54) Novel compounds

(76) Inventors: Goran Magnusson, Lund (SE); Pia Magnusson Hall, Lund (SE); Mattias Belting, Del Mar, CA (US); Niklas Falk, Lund (SE); Lars-Ake Fransson, Lund (SE); Katrin Mani, Lund (SE)

Correspondence Address: BURNS DOANE SWECKER & MATHIS L L P, POST OFFICE BOX 1404, ALEXANDRIA, VA, 22313-1404, US

(21) Appl. No.: 10/182,135

(22) PCT Filed: Jan. 30, 2001
(86) PCT No.: PCT/SE01/00167

(30) Foreign Application Priority Data
Jan. 31, 2000 (SE) 0000303-8

(51) Int. Cl. A61K 31/7052; A61K 31/704; A61K 31/517; A61K 31/4709; A61K 31/35

(52) U.S. Cl. 514/25; 514/33; 514/459; 514/266.24; 514/314; 514/460; 514/312

(57) ABSTRACT

The present invention relates to combinations of xylose compounds with other pharmaceutically active compounds, to pharmaceutical compositions comprising said combinations, as well as to use of these combinations for the manufacture of a medicament for treatment of proliferative disorders. In another aspect, the present invention relates to novel xylose compounds, to pharmaceutical compositions comprising said compounds, and to use of these compounds for the manufacture of a medicament or the treatment of proliferative disorders.

### FIELD OF THE INVENTION

The present invention relates to combinations of xylose compounds with other pharmaceutically active compounds, to pharmaceutical compositions comprising said combinations, as well as to use of these combinations for the manufacture of a medicament for the treatment of proliferative disorders. In another aspect, the present invention relates to novel xylose compounds, to pharmaceutical compositions comprising said compounds, and to use of these compounds for the manufacture of a medicament for the treatment of proliferative disorders.

### BACKGROUND OF THE INVENTION

It is well known that new improved treatment of proliferative disorders, particularly turnout diseases, is an ongoing demand in the field of medicine. Accordingly, a very large number of approaches have been disclosed in order to solve this general problem. One of them is the continuous development of new anti-proliferative drugs. Typical such drugs are alkylating agents, e g Alkeran®, antimetabolites, e g methotrexate, pyrimidine analogues, e g fluorouracil, mitosis inhibitors, e g vincristine, podophyllotoxin derivatives, e g Vepesid®, and taxanes, e g taxol. Other examples are α-difluoromethylornithine (DFMO), cisplatin and suramin. A further example is β-D-xylosides having an estrogen aglycone, as disclosed in U.S. Pat. No. 5,104,856, the entire teachings of which are enclosed herein by reference.

### DISCLOSURE OF THE INVENTION

The invention is based on a specific group of xylose compounds which unexpectedly provide a synergistic anti-proliferative effect when utilised in combination with specific groups of anti-tumour agents, as will be further specified hereinbelow. The group of xylose compounds referred to comprises known as well as novel compounds, and the anti-tumour agents referred to are generally known, non-xylose compounds which yield an anti-proliferative effect.

More specifically, the present invention is based on a composition comprising an anti-tumor agent and a glycoside of xylose having an O- or S-glycosidically linked aglycone, where the aglycone contains at least one aromatic ring. Preferably, said aglycone contains at least two carbocyclic structures, of which at least one is aromatic, where said at least two carbocyclic structures are optionally condensed to one carbocyclic structure and/or contain at least one heteroatom selected from O, N and S.

More specifically still, the present invention relates to an anti-proliferatively active composition, comprising

a) at least one compound having the general formula (I)

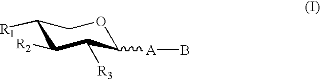

wherein

A is O;

B is selected from naphthyl, naphthylalkyl, anthracenyl, anthracenylalkyl, benzo[a]anthracenyl, benzo[a]anthracenylalkyl, benzo[b]anthracenyl, benzo[b]anthracenylalkyl, benzo[c]anthracenyl, benzo[c]anthracenylalkyl, phenanthrenyl, phenanthrenylalkyl, benzo[a]phenantrenyl, benzo[a]phenantrenylalkyl, benzo[b]phenantrenyl, benzo[b]phenantrenylalkyl, benzo[c]phenantrenyl, benzo[c]phenantrenylalkyl, biphenyl, biphenylalkyl, quinolinyl, quinzolinyl and quinoxalinyl; B optionally being substituted with at least one group selected from OY, F, Cl, Br, I, NO₂, CF₃, COOH, NH₃ alkyl, NHC(O)-alkyl and C(O)O-alkyl;

R₁-R₃ are independently selected from F, NHAc and OY;

Y is independently selected from H, C₂₋₆-acyl, alkyl and aralkyl, where the alkyl group has 1-6 carbon atoms;

and pharmaceutically acceptable salts thereof, in combination with

b) at least one anti-tumour agent selected from the group consisting of polyamine synthesis inhibitor, polyamine cellular uptake inhibitor, polyamine degradation promotor and epoxygenase inducer;

said combination of compound(s) a) and anti-tumour agent(s) b) being selected such that a synergistic anti-proliferative activity is accomplished.

In some embodiments of the invention, alkyl in connection with B in compound(s) a) has 1-6 carbon atoms.

Compound(s) a) preferably comprise(s) at least one β-glycoside. Furthermore, in preferred embodiments, compound(s) a) comprises) at least one D-xyloside.

In certain embodiments of the invention, B in compound(s) a) is naphthyl substituted with at least one OH group. Among these embodiments, those were B is naphthyl substituted with two OH groups, especially selected from 5,6-dihydroxynaphthyl, 6,7-dihydroxynaphthyl, 1,4-dihydroxynaphthyl and 5,8-dihydroxynaphthyl are preferred. Thus, of those embodiments of compound(s) a) where B is naphthyl with two OR groups, 5,6-dihydroxynaphthyl-β-D-xylopyranoside, 6,7-dihydroxynaphthyl-β-D-xylopyranoside, 1,4-dihydroxynaphthyl-β-D-xylopyranoside and 5,8-dihydroxynaphthyl-β-D-xylopyranoside are preferred.

However, in another preferred embodiment, B in compound(s) a) is 6-hydroxynaphthyl, and preferred compound(s) a) comprise(s) 6-hydroxy-2-naphthalenyl-β-D-xylopyranoside, hereinafter referred to as Xyl-2-Nap-6-OH.

Said anti-tumor agent(s) b) comprise(s) an agent which inhibits synthesis or cellular uptake of polyamines and/or promotes polyamine degradation or epoxygenase activity. In embodiments of the invention, the polyamine synthesis inhibitor can be α-difluoromethylornithine (DFMO). The polyamine cellular uptake inhibitor is preferably suramin. In certain embodiments, the polyamine degradation promotor is a nitric oxide donor. A number of suitable nitric oxide donors are known, and they are well exemplified in e g WO 96/35416, the teachings of which are incorporated herein by reference. The nitric oxide donor is preferably selected from nitroglycerin, S-nitrosothiols and a sydnoimine, such as molsidomine or linsidomine. The epoxygenase inducer is preferably naphtho-flavone.

In one embodiment of the invention, the anti-tumour agent(s) b) is(are) selected from suramin and DFMO. Preferably, anti-tumour agent b) is a combination of both suramin and DFMO.

Thus, one preferred combination of compounds(s) a) and anti-tumour agent(s) b) according to the invention is Xyl-2-Nap-6-OH together with ore of suramin and DFMO, or with a combination of both suramin and DFMO.

Furthermore, the present invention relates to a combination of compound(s) a) and anti-tumour agent(s) b) as set forth above for use as a pharmaceutical.

Accordingly, the present invention also relates to a pharmaceutical composition comprising a combination of compound(s) a) and anti-tumour agent(s) b) as set forth above as active ingredient in association with a pharmaceutically acceptable adjuvant, diluent or carrier.

In addition, the present invention relates to the use of a combination of compound(s) a) and anti-tumour agent(s) b) as set forth above for the manufacture of a medicament for treatment of proliferative disorders, particularly tumor diseases. As examples of such proliferative disorders, mention can be made of lung cancer, e g adenocarcinoma of the lung, and small cell carcinoma of the lung; stomach cancer, e g carcinoma of the stomach; colon cancer, e g adenocarcinoma of the colon; liver cancer, e g hepatocellular carcinoma; prostata carcinoma; breast cancer, e g breast carcinoma; malignant melanoma; and brain tumors, e g astrocytoma, glioma, and meningioma.

The present invention is also concerned with a method for treatment of proliferative disorders, particularly tumor diseases, wherein said method comprises administering of a therapeutically effective amount of a combination of compound(s) a) and anti-tumour agent(s) b) as set forth above to a human or animal patient.

The typical dosages of said compound(s) a) and anti-tumour agent(s) b) vary within a wide range and will depend on various factors, such as the particular requirement of each receiving individual and the route or administration. However, the dosages are generally within the range of 0.001-100 mg/kg body weight for a) and b) each.

The synergism concept of the present invention may be further illustrated by the following Scheme 1:

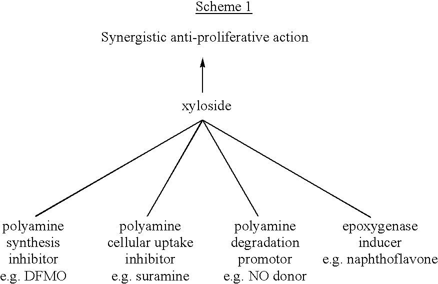

In a different aspect, the present invention also relates to novel compounds having the general formula (I)

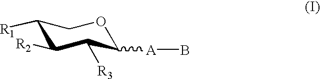

wherein

A is selected from O and S;

B is selected from naphthyl, naphthylalkyl, anthracenyl, anthracenylalkyl, benzo[a]anthracenyl, benzo[a]anthracenylalkyl, benzo [b]anthracenyl, benzo[b]anthracenylalkyl, benzo[c]anthracenyl, benzo[c]anthracenylalkyl, phenanthrenyl, phenanthrenylalkyl, benzo[a]phenantrenyl, benzo[a]phenantrenylalkyl, benzo[b]phenantrenyl, benzo[b]phenantrenylalkyl, benzo[c]phenantrenyl, benzo[c]phenantrenylalkyl, biphenyl, biphenylalkyl, quinolinyl, quinazolinyl and quinoxalinyl or conventional derivative thereof; B optionally being substituted with at least one group selected from OY, F, Cl, Br, I, NO₂. CF₃, COOH, NH₂, alkyl, NHC(O)-alkyl and C(O)O-alkyl;

and pharmaceutically acceptable salts thereof;

R₁-R₃ are independently selected from F, NHAc and OY with the proviso that no more than two of R₁-R₃ are F or NHAc;

Y is independently selected from H, C₂₋₆-acyl, alkyl and aralkyl, where the alkyl group has 1-6 carbon atoms;

with the proviso that B is not 1-naphthyl, 2-naphthyl, 2-(6-ethoxy)naphthyl, 2-(6-butoxy)naphthyl, 2-(6-hydroxy)naphthyl, 2-(6-bromo)naphthyl, 2-naphthalenylmethyl, 9-phenanthrenyl, 1-anthracenyl, 6-quinolinyl or 4-biphenyl.

As to preferred embodiments cc the various groups and substituents of the novel compounds according to the invention, these are the same as those described earlier in regard to compound(s) a) in sea combination of compound(s) a) and anti-tumour agent(s) b) However, the provisos regarding the nature of B and R₁-R₃ are still applicable.

The-novel compounds as set forth above are, per se, useful as active ingredients in pharmaceutical compositions, for manufacture of medicaments against proliferative disorders, and for methods of treatment of proliferative disorders, to the same extent as the combinations of compound(s) a) and anti-tumour agent(s) b) described earlier.

The following examples are intended as a non-limiting illustration of the invention.

### BRIEF DESCRIPTION OF THE FIGURES

FIG. 1 is a three-dimensional diagram showing the effect of various concentrations of Xyl-2-Nap-6-OH, suramin and the combination of Xyl-2-Nap-6-OH with suramin on growth of (A) human lung fibroblasts (HFL-1 cells), and (B) transformed human vascular endothelial cells (ECV cells).

FIG. 2 is a diagram showing the effect of 0.05 mM Xyl-2-Nap-6-OH, 0.2 mM suramin and the combination of 0.05 mM Xyl-2-Nap-6-OH with 0.2 mM suramin on growth of (A) transformed human vascular endothelial cells (ECV cells), and (B) human lung fibroblasts (HFL-1 cells) FIG. 2A also shows the effect of the combination of 0.5 mM Xyl-2-Nap-6-OH with 5 mM DFMO and 0.2 mM suramin on growth of ECV cells.

FIG. 3 is a diagram showing the effect of various concentrations of Xyl-2-Nap-6-OH, 5 mM DFMO and the combination of Xyl-2-Nap-6-OH with 5 mM DFMO on growth of transformed human vascular endothelial cells (ECV cells).

### EXAMPLES

Preparation of Novel Xylosides

¹H NMR-spectra were recorded at 400 MHz proton frequency, using CD₃OD as solvent and CH₃OD (δ 3.34) as internal standard. TLC analyses were performed with Merck SiO₂ 60 F₂₅₆ precoated aluminium sheets with visualisation by UV light, charring with H₂SO₄ (10% in water) with 0.2% orcinol. CH₃CN (Merck) was dried by distillation from CaH₂. BF₃Et₂O (Acros) was used as received.

All β-D-xylosides were prepared by using well-established trichloroacetimidate methodology. A typical example is depicted in Scheme 2:

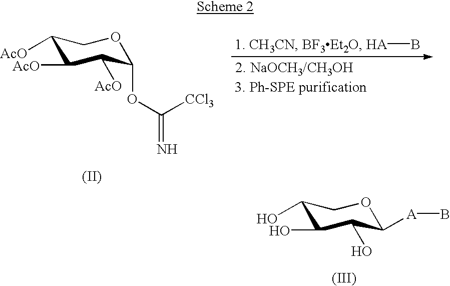

The Ph-solid phase extraction procedure (Ph-SPE; Thurman E M, Mills M S (eds)(1998) Solid Phase Extraction. Wiley-Interscience, New York) is further simplified by using an excess of donor (II), thereby ensuring complete consumption of the acceptor HA-B. The generally both hydrophobic and aromatic nature of the aglycone A-B provides an easy separation of the product (III) from the water soluble other constituents of the reaction mixture. A typical work-up of the reaction mixture is performed by adding water to the reaction mixture to such an extent that the organic solvent constitutes less than 10 percent by volume of the total solution to be purified. This solution is then applied onto a conditioned Ph-silica plug, which is washed with water, after which pure (III) is eluted from the plug by using methanol The Ph-silica plug can be reused, and it is easily reconditioned for a new purification. In fact, a product (III) pure enough for biological testing can be prepared in less than 1 h by using the methodology of Scheme 2.

As a typical non-limiting example, the detailed preparation of 6-methoxy-2-naphthalenyl-β-D-xylopyranoside (i.e. A is O, whereas B is 6-methoxynaphtyl; compound IV) is exemplified below;

To a stirred solution of trichloroacetimidoyl-2,3,4-tri-O-acetyl-β-D-xylopyranoside (50 mg, 0.12 mmol) and 6-methoxy-2-naphthol (12.8 mg, 0.0741 mmol) in acetonitrile (CH₃CN, 1 ml) was added boron trifluoride etherate (BF₃ Et₂O, 0.001 ml, 0.008 mmol) at room temperature After 30 minutes, a 1 M solution of sodium methoxide (NaOCH₃) in methanol (CH₃OH, 0.1 ml) was added. After 5 minutes, the reaction mixture was diluted by addition of water (3 ml). The resulting solution was applied to a Ph-SiO₂ column (IST Isolute™ SPE column, 500 mg PH, item #360-0050-B, commercially available from Sorbent AB, Box 8039, 42108 Västra Frölunda, Sweden), which had previously been conditioned by elution with acetonitrile:water 1:3. The column was then eluted with water (5 ml) followed by methanol (5 ml). After concentration of the methanol solution, the desired product IV was obtained as a white powder (15.8 mg, 0.0516 mmol) in 70% yield. Compound data: ¹H NMR (400 MHz; CD₃OD) δ 7.74-7.66 (m, 2H), 7.41-7.36 (m, 1H), 7.28-7.23 (m, 1H), 7.22-7.18 (m, 1H), 7.15-7.09 (m, 1H), 4.99 (d, J=7.22 Hz, 1H), 3.99 (dd, J=11.30, 5.28 Hz, 1H), 3.90 (s, 3H), 3.68-3.59 (m, 1H), 3.54-3.41 (m, 3H); HRMS FAB MS calculated for C₁₆H₁₈NaO₆⁺ (M+Na⁺): 329.1001; found: 329.1000.

The following novel compounds were also prepared by the above reaction protocol:

7-Methoxy-2-naphthalenyl-β-D-xylopyranoside (V) Compound data: ¹H NMR (400 MHz; CD₃OD) δ 7.74-7.68 (m, 2H), 7.38-7.36 (m, 1H), 7.27-7.10 (m, 2H), 7.04-7.00 (m, 1H) 5.04 (d, J=7.27 Hz, 1H). 4.00 (dd, J=7.62, 3.71 Hz, 1H), 3.92 (s, 3H), 3.70-3.60 (m, 2H), 3.55-3.44 (m, 2H); HRMS FAB MS calculated for C₁₆H₁₈NaO₆⁺ (M+Na⁺): 329.1001; found: 329.002.

[1,1′-Biphenyl]-3-yl-β-D-xylopyranoside (VI) Compound data: ¹H NMR (400 MHz; CD₃OD) δ 7.64-7.06 (m, 9H), 4.96 (d, J=7.28 Hz, 1H), 3.97 (dd, J=11.35, 5.29 Hz, 1H), 3.70-3.58 (m, 1H), 3.51-3.37 (m, 3H): HRMS FAB MS calculated for C₁₇H₁₈NaO₅⁺ (M+Na⁺): 325.1052; found: 325.1051.

1-Naphthalenylmethyl-β-D-xylopyranoside (VII) Compound data: ¹H NMR (400 MHz; CD₃OD) δ 8.22-8.18 (m, 1H), 7.90-7.80 (m, 2H), 7.59-7.41 (m, 4H), 5.36 (d, J=11.54 Hz, 1H), 5.02 (d, J=11.38 Hz, 1H), 4.41 (d, J=7.43 Hz, 1H), 3.95 (dd, J=11.46, 5.37 Hz, 1H), 3.53 (ddd, J=10.27, 8.65, 5.38 Hz, 1H), 3.34-3.22 (m, 3H); HRMS FAB MS calculated for C₁₆H₁₈NaO₅⁺ (M+Na⁺): 313.1052; found: 313.1046.

[1,1′-Biphenyl]-4-ylmethyl-β-D-xylopyranoside (VIII) Compound data: ¹H NMR (400 MHz; CD₃OD) δ 7.63-7.58 (m, 4H), 7.50-7.46 (m, 2H), 7.45-7.40 (m, 2H), 7.38-7.30 (m, 1H), 4.93-4.83 (m, 1H), 4.68 (d, J=11.90 Hz, 1H), 4.33 (d, J=7.34 Hz, 1H), 3.91 (dd, J=11.41, 5.37 Hz, 1H), 3.51 (ddd, J=10.10, 8.52, 5.36 Hz, 1H), 3.34-3.18 (m, 3H); HRMS FAB MS calculated for C₁₈H₂₀NaO₅⁺ (M+Na⁺): 339.1208; found: 339.1209.

As further non-limiting examples of an acceptor HA-B suitable for use in providing the present novel xylosides mention can be made of [1,1′-biphenyl]-4,4′-diimethanol, 4′-hydroxy-[1,1′-biphenyl]-4-methanol, 4′-bromo-[1,1′-biphenyl]-4-ol, 6-bromo-2-naphthalenemethanol, 6-hydroxy-2-naphthalenemethanol, 9,10-anthracenediol, 9-anthracenol, 4,5-phenanthrenediol, benzo[c]phenanthren-1-ol, 9-anthracenemethanol, 1-anthracenemethanol, 2-anthracenemethanol, 6-bromo-2-phenanthrenemethanol, 9,10-dihydro-2,7-phenanthrenedimethanol, 1,8,9-anthracenetriol, 2-amino-6-naphthol, 1-amino-5-naphthol, 4′-amino-[1,1′-biphenyl]-4-ol, naphtho[2,3-d]-1,3-dioxol-6-ol, [2,2′-binaphthalene]-6,6′-diol, 1,4-naphthalenediol, or 4-hydroxy-1-naphthalenemethanol.

The exemplified procedure is easily utilised, and if necessary also modified, by a person skilled in the art, should it be desired to prepare any one of the novel xylosides of the present invention which are not explicitly disclosed herein. If deemed suitable, other conventional glycosylation may of course be used in the preparation of the present novel xylosides (Khan S H, O'Neill R A (eds) (1996) Modern methods in carbohydrate synthesis. Harwood Academic Publishers, Amsterdam; Hanessian S (ed) (1997) Preparative carbohydrate chemistry. Marcel Dekker, New York).

The already known b-D-xylopyranosides (vide supra) preferably utilised in the practising of the present invention were manufactured according to standard literature procedures (Xyl-naphtOH) Cancer Res (1998), 58(6), 1099-1104. (Xyl-naphtBr) Fiziol Rast (Moscow) (1978), 25(6), 1281-7.

Cellular Growth Assays

Human embryonic lung fibroblasts (HFL-1 cells) and transformed endothelial cells (ECV cells) were obtained. Monolayer cultures were maintained on plastic in Eagle's MEM (Life Technologies, Ltd, Renfrewshire, UK). The medium was supplemented with 10% FCS (In Vitro AB, Stockholm, Sweden), 2 mM L-glutamine (ICN Biochemicals), penicillin (100 units/ml) and streptomycin (100 μg/ml) Cells were regularly checked for mycoplasma using GEN-PROBE Rapid detection system (Skafte & Claesson, Mölndal, Sweden).

The growth assay procedure has been described in detail in Westergren-Thorsson et al (1991), J Cell Physiol 147:523-530. In brief, cells were harvested by trypsinisation and seeded into 96-well microculture plates at 3000-5000 cells/well in Ham's F-12 medium (Sigma) supplemented with insulin (10 ng/ml), transferrin (25 ng/ml) and 10% FCS. After 4 h of plating, the cells were serum-starved for 24 h. Cells were then allowed to proliferate, supported by 10 ng/ml of epidermal growth factor (Genzyme, Cambridge, Mass., USA), in the presence or absence of various concentrations of the anti-proliferatively active compositions according to the present invention. Controls without growth factor and controls with different concentrations of DMSO were included. After 72, 96 and 120 h of growth, cells were fixed in glutaraldehyde, and cell nuclei were stained with crystal violet (Merck, Darmstadt, Germany). After washing and cell lysis for 24 h in 1% Triton X-100, the amount of bound dye was measured at 595 nm in a microplate photometer (Titertek multiscan). Results were corrected for DMSO effects.

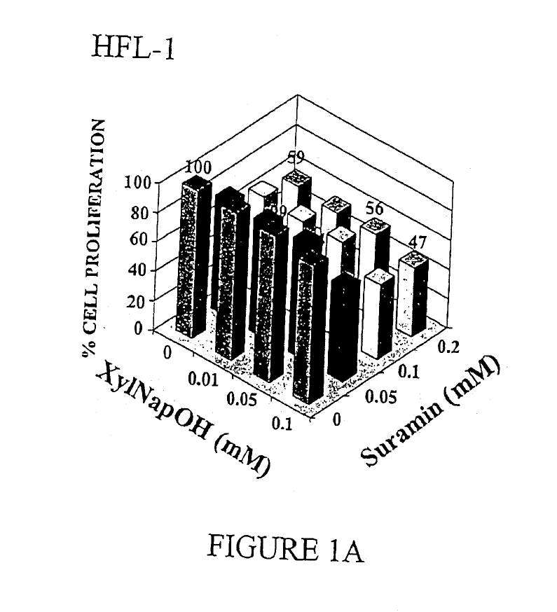

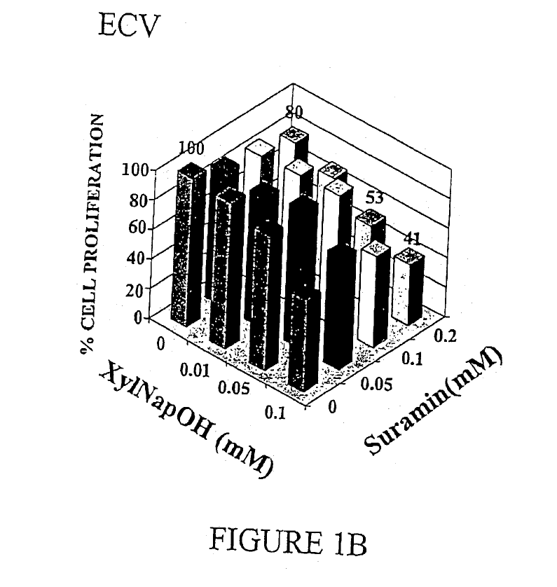

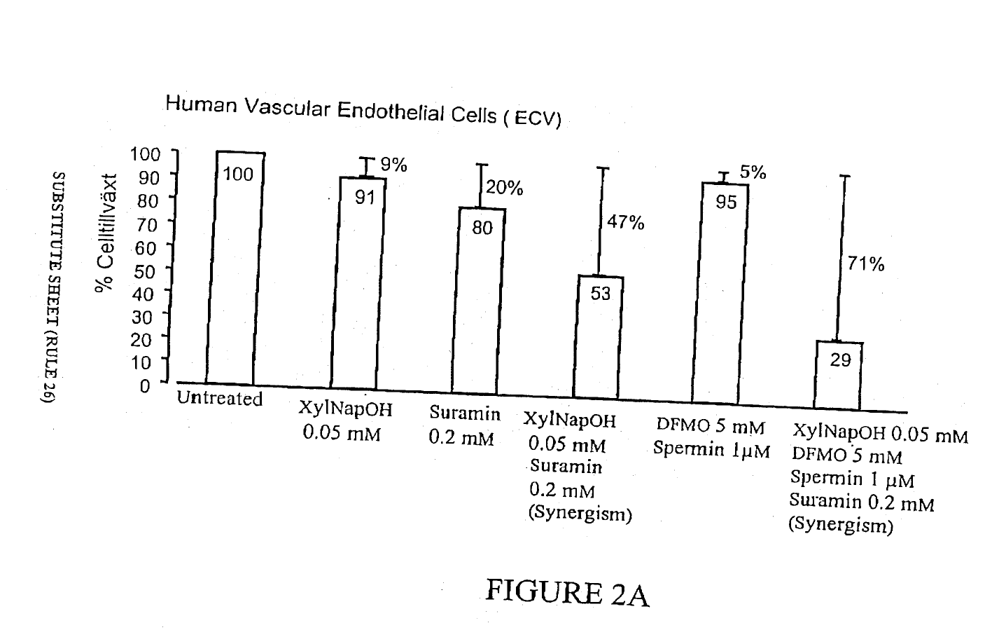

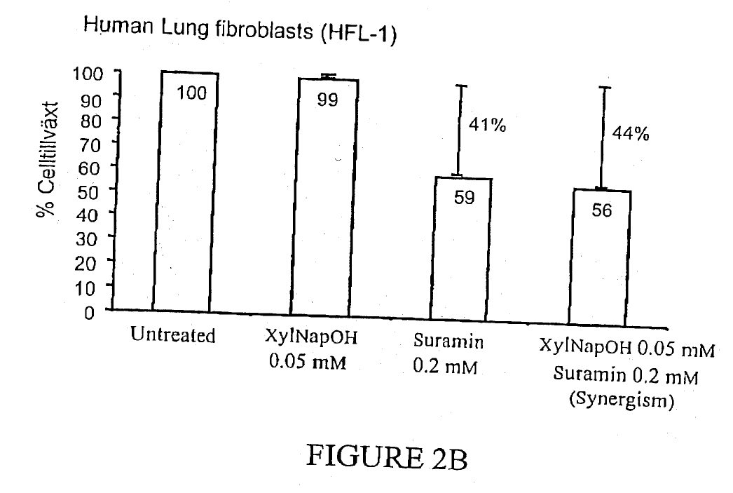

Referring to FIGS. 1 and 2, the results of treating cells with a combination of Xyl-2-Nap-6-OH with suramin are shown. Proliferation of untreated cells is used as reference. As is evident from FIGS. 1B and 2A, treatment of transformed endothelial cells (ECV cells) with 0.05 mM of Xyl-2-Nap-6-OH inhibits growth of ECV cells by 9%, while treatment with 0.2 mM of suramin inhibits their growth by 20%. The combination of 0.05 mM of Xyl-2-Nap-6-OH with 0.2 mM of suramin gives a synergistic effect of 47% inhibition, rather than an additive effect of 29% that might be expected. This synergistic effect could not be seen in an analogous experiment conducted on normal, untransformed HFL-1 cells (FIGS. 1A and 2B). Furthermore, ECV cells that have been made dependent on 1 μM spermin (a polyamine) through treatment with 5 mM DFMO were growth inhibited by 5%. When spermin dependent cells were treated with 0.05 mM Xyl-2-Nap-6-OH in combination with 0.2 mM suramin a synergistic effect of 71% inhibition was observed, rather than an additive effect of 52% (FIG. 2A).

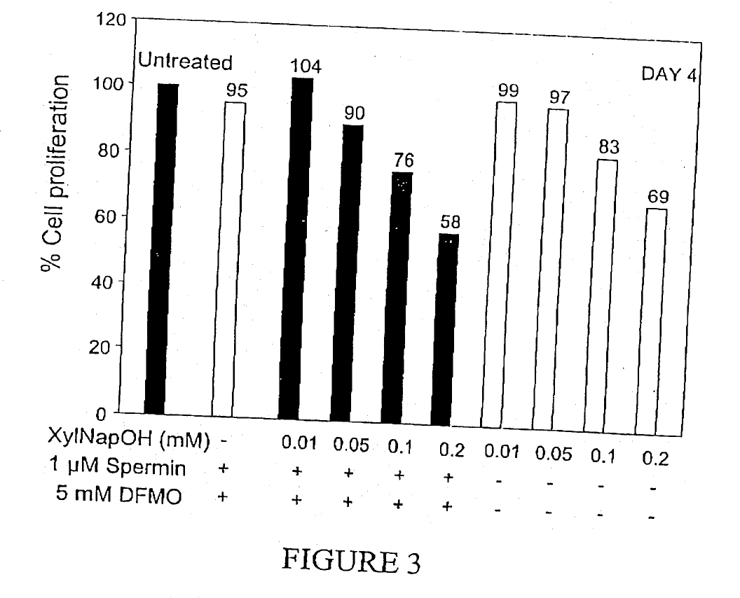

Referring now to FIG. 3, the results of treating ECV cells with a combination of Xyl-2-Nap-6-OH with DFMO are shown. As in the previous experiment, proliferation of untreated cells is used as reference. As is evident from the diagram, ECV cells that have been made dependent on 1 μM spermin (a polyamine) through treatment with 5 mM DFMO exhibit an increased sensitivity towards Xyl-2-Nap-6-OH. Treating the cells with the combination of 5 EM DFMO and 0.2 mM of Xyl-2-Nap-6-OH results in a cell proliferation of 58; as compared to untreated cells, i e an inhibition of growth of 42%. Treatment with Xyl-2-Nap-6-OH alone results in 31% inhibition of growth, and treatment with DFMO only (second bar from left in the diagram) results in a mere 5% inhibition. Since the expected additive effect thus is less than the actual effect, treatment with the combination of DFMO and Xyl-2-Nap-6-OH results in a synergistic inhibition of the proliferation of transformed cells.

1. An anti-proliferatively active composition, comprising
a) at least one compound having the general formula (I) 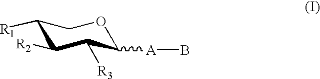
wherein
A is O;
B is selected from naphthyl, naphthylalkyl, anthracenyl, anthracenylalkyl, benzo[a]anthracenyl, benzo[a]anthracenylalkyl, benzo[b]anthracenyl, benzo[b]anthracenylalkyl, benzo[c]anthracenyl, benzo[c]anthracenylalkyl, phenanthrenyl, phenanthrenylalkyl, benzo[a]phenantrenyl, benzo[a]phenantrenylalkyl, benzo[b]phenantrenyl, benzo[b]phenantrenylalkyl, benzo[c]phenantrenyl, benzo[c]phenantrenylalkyl, biphenyl, biphenylalkyl, quinolinyl, quinazolinyl and quinoxalinyl; B optionally being substituted with at least one group selected from OY, F, Cl, Br, I, NO₂. CF₃, COOH, NH₂, alkyl, NHC(O)-alkyl and C(O)O-alkyl;
R₁-R₃ are independently selected from F, NHAc and OY;
Y is independently selected from H, C₂₋₆-acyl, alkyl and aralkyl, where the alkyl group has 1-6 carbon atoms; arid pharmaceutically acceptable salts thereof,
in combination with
b) at least one anti-tumour agent selected from the group consisting of polyamine synthesis inhibitor, polyamine cellular uptake inhibitor, polyamine degradation promotor and epoxygenase inducer;
said combination of compound(s) a) and anti-tumour agent(s) b) being selected such that a synergistic anti-proliferative activity is accomplished.

2. A composition according to claim 1, wherein alkyl at each occurrence in connection with B has 1-6 carbon atoms.

3. A composition according to any one of the preceding claims, wherein a) comprises at least one β-glycoside.

4. A composition according to any one of the preceding claims, wherein a) comprises at least one D-xyloside.

5. A composition according to any one of the preceding claims, wherein B is naphthyl which is substituted with at least one OH group.

6. A composition according to claim 5, wherein said substituted naphthyl group is 6-hydroxynaphthyl.

7. A composition according to any one of the preceding claims, wherein a) comprises 6-hydroxy-2-naphthalenyl-β-D-xylopyranoside.

8. A composition according to claim 5, wherein said naphthyl group is substituted with two OH groups.

9. A composition according to claim 8, wherein said substituted naphthyl group is chosen from 5,6-dihydroxynaphthyl, 6,7-dihydroxynaphthyl, 1,4-dihydroxynaphthyl and 5,8-dihydroxynaphthyl.

10. A composition according to claim 9, wherein a) comprises a β-D-xylopyranoside selected From 5,6-dihydroxynaphthyl-β-D-xylopyranoside, 6,7-dihydroxynaphthyl-β-D-xylopyranoside, 1,4-dihydroxynaphthyl-β-D-xylopyranoside and 5,8-dihydroxynaphthyl-β-D-xylopyranoside.

11. A composition according to any one of the preceding claims, wherein said polyamine synthesis inhibitor is α-difluoromethylornithine.

12. A composition according to any one of the preceding claims, wherein said polyamine cellular uptake inhibitor is suramin.

13. A composition according to any one of the preceding claims, wherein said polyamine degradation promotor is a nitric oxide donor.

14. A composition according to claim 13, wherein said nitric oxide donor is selected from nitroglycerin, S-nitrosothiols and a sydnoimine.

15. A composition according to claim 14, wherein said sydnoimine is selected from molsidomine and linsidomine.

16. A composition according to any one of the preceding claims, wherein said epoxygenase inducer is naphthoflavone.

17. A composition according to any one of claims 1-7 and 12, wherein a) is 6-hydroxy-2-naphthalenyl-β-D-xylopyranoside and b) is suramin.

18. A composition according to any one o. Claims 1-7 and 11, wherein a) is 6-hydroxy-2-naphthalenyl-β-D-xylopyranoside and b) is α-difluoromethylornithine.

19. A composition according to any one of claims 1-7 and 11-12, wherein a) is 6-hydroxy-2-naphthalenyl-β-D-xylopyranoside and h) is a combination of suramin and α-difluoromethylornithine.

20. A pharmaceutical composition, comprising a combination of compound(s) a) and anti-tumour agent(s) b) as defined in any one of the preceding claims as an active ingredient in association with a pharmaceutically acceptable adjuvant, diluent or carrier.

21. A pharmaceutical composition according to claim 20, in which said combination is present in an amount such that a dose, for each of compound(s) a) and anti-tumour agent(s) b), in the range of 0.001-100 mg/kg body weight is obtained upon administration.

22. Use of a pharmaceutical composition according to any one of claims 20-21 for the manufacture of a medicament for treatment of a proliferative disorder.

23. Use according to claim 22, wherein said proliferative disorder is a tumour disease.

24. Use according to claim 23, wherein said tumour disease is lung cancer, stomach cancer, colon cancer, liver cancer, prostata carcinoma, breast cancer or a brain tumour.

25. A method for treatment of proliferative disorders, particularly tumour diseases, wherein said method comprises administering of a therapeutically effective amount of a combination of compound(s) a) and anti-tumour agent(s) b) according to any one of claims 1-21 to a human or animal patient.

26. A method according to claim 25, wherein the administered doses of compound(s) a) and anti-tumour agent(s) b) of said combination is within the range of 0.001-100 mg/kg body weight each.
